SEQUENCE LISTING (2 sequences)

SEQ ID NO: 1 - protein, 539 aa, Homo sapiens
  MISC_FEATURE (1)..(539): Lck protein (GenBank Accession No. AAH13200)
  MISC_FEATURE (65)..(118): Src homology 3 (SH3) domain
  MISC_FEATURE (123)..(223): Src homology 2 (SH2) domain
  MISC_FEATURE (237)..(503): Catalytic domain
MGCGCSSHPEDDWMENIDVCENCHYPIVPLDGKGTLLIRNGSEVRDPLVTYEGSNPPASPLQDNLVIALHSYEPSHDGDLGFEKGEQLRILEQSGEWWKAQSLTTGQEGFIPFNFVAKANSLEPEPWFFKNLSRKDAERQLLAPGNTHGSFLIRESESTAGSFSLSVRDFDQNQGEVVKHYKIRNLDNGGFYISPRITFPGLHELVRHYTNASDGLCTRLSRPCQTQKPQKPWWEDEWEVPRETLKLVERLGAGQFGEVWMGYYNGHTKVAVKSLKQGSMSPDAFLAEANLMKQLQHQRLVRLYAVVTQEPIYIITEYMENDTLLDSQLEEKGLGASPWGNLGQQLLLLPTGSLVDFLKTPSGIKLTINKLLDMAAQIAEGMAFIEERNYIHRDLRAANILVSDTLSCKIADFGLARLIEDNEYTAREGAKFPIKWTAPEAINYGTFTIKSDVWSFGILLTEIVTHGRIPYPGMTNPEVIQNLERGYRMVRPDNCPEELYQLMRLCWKERPEDRPTFDYLRSVLEDFFTATEGQYQPQP

SEQ ID NO: 2 - protein, 363 aa, Homo sapiens
  MISC_FEATURE (1)..(363): lymphocyte-specific protein tyrosine kinase (Lck) (GenBank Accession No. AAC50287.1)
  MISC_FEATURE (65)..(118): Src homology 3 (SH3) domain
  MISC_FEATURE (123)..(223): Src homology 2 (SH2) domain
  MISC_FEATURE (237)..(348): catalytic domain
MGCGCSSHPEDDWMENIDVCENCHYPIVPLDGKGTLLIRNGSEVRDPLVTYEGSNPPASPLQDNLVIALHSYEPSHDGDLGFEKGEQLRILEQSGEWWKAQSLTTGQEGFIPFNFVAKANSLEPEPWFFKNLSRKDAERQLLAPGNTHGSFLIRESESTAGSFSLSVRDFDQNQGEVVKHYKIRNLDNGGFYISPRITFPGLHELVRHYTNASDGLCTRLSRPCQTQKPQKPWWEDEWEVPRETLKLVERLGAGQFGEVWMGYYNGHTKVAVKSLKQGSMSPDAFLAEANLMKQLQHQRLVRLYAVVTQEPIYIITEYMENGSLVDFLKTPSGIKLTINKLLDMAAQVRRLGRGAGQGNRPVT

(19) United States
(12) Patent Application Publication
(10) Pub. No.: US 2021/0332151 A1
(43) Pub. Date: Oct. 28, 2021

(54) Lck AS A THERAPEUTIC TARGET IN IDIOPATHIC PULMONARY FIBROSIS

(71) Applicant: Temple University - Of The Commonwealth System Of Higher Education, Philadelphia, PA (US)

(72) Inventors: Beata KOSMIDER, Havertown, PA (US); Karim BAHMED, Havertown, PA (US)

(21) Appl. No.: 17/240,596

(22) Filed: Apr. 26, 2021

Related U.S. Application Data
(60) Provisional application No. 63/015,844, filed on Apr. 27, 2020.

(51) Int. Cl. C07K 16/40; A61P 11/00; A61K 39/395; A61K 31/4412; A61K 31/496; A61K 31/52; A61K 31/198; G01N 33/68

(52) U.S. Cl. CPC C07K 16/40; A61P 11/00; A61K 39/3955; A61K 31/4412; A61K 48/00; A61K 31/52; A61K 31/198; G01N 33/6893; A61K 31/496

(57) ABSTRACT

This disclosures provides methods of treatment of idiopathic pulmonary fibrosis (IPF) with an intrabody. Methods of screening for IPF are also provided. Methods for inhibiting fibroblast proliferation, fibroblast differentiation, and/or fibroblast activation of T cells with an intrabody are also provided.

### CROSS REFERENCE TO RELATED APPLICATIONS

The application claims the benefit of the filing date of U.S. Provisional Patent Application No. 63/015,844, filed Apr. 27, 2020, the entire disclosure of which is incorporated herein by reference.

### SEQUENCE LISTING

The instant application contains a Sequence Listing which has been submitted electronically in ASCII format and is hereby incorporated by reference in its entirety. Said ASCII copy, created on Apr. 26, 2021, is named 035926-0505-000004_SL.txt and is 8,894 bytes in size.

### FIELD OF THE INVENTION

This disclosure relates to a new therapeutic agents for the treatment of idiopathic pulmonary fibrosis.

### BACKGROUND

Idiopathic pulmonary fibrosis (IPF) is a progressive, debilitating and life threatening disease of unknown etiology and has no cure. The median survival of patients with IPF is 2 to 3 years. Lung transplantation is the only intervention shown to increase life expectancy for IPF patients. However, lung transplantation is associated with high mortality. IPF affects 3 million people worldwide and 130,000 people in the United States (Glassberg, 2019, Am J Manag Care 25, S195-S203).

### SUMMARY OF THE INVENTION

The disclosure is based on the discovery that there is a high lymphocyte-specific protein tyrosine kinase (Lck) expression in lung tissue of patients having idiopathic pulmonary fibrosis (IPF). The increased Lck is believed to contribute to increased secretion of profibrotic factors by CD4+ and CD8+ T cells that inhibit re-epithelization in IPF.

### BRIEF DESCRIPTION OF THE FIGURES

The accompanying drawings, which are included to provide a further understanding of the disclosure and are incorporated in and constitute a part of this specification, illustrate exemplary embodiments of the disclosure, and together with the description serve to explain the principles of the disclosure.

FIGS. 1A and 1B depict data relating to lymphocyte-specific protein tyrosine kinase (Lck) expression in lung tissue in IPF. Lung tissue was obtained from controls (C) and IPF patients (IPF). FIG. 1A is an image of a Western blot and FIG. 1B is a histogram graph of the relative Lck expression (relative to GAPDH expression) in lung tissue from controls and from IPF patients. N=3 lungs per group. *: p<0.05.

### DEFINITIONS

Unless defined otherwise, all technical and scientific terms used herein have the same meaning as commonly understood by one of ordinary skill in the art to which the invention pertains.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element. Thus, recitation of “a cell”, for example, includes a plurality of the cells of the same type.

“About” as used herein when referring to a measurable value such as an amount, a temporal duration, and the like, is meant to encompass variations of +/−20% or +/−10%, more preferably +/−5%, even more preferably +/−1%, and still more preferably +/−0.1% from the specified value, as such variations are appropriate to perform the disclosed methods.

An “effective amount” as used herein, means an amount of a therapeutic compound, when administered to a patient suffering from IPF, provides a therapeutic benefit in alleviating one or more manifestations of IPF. It is understood, however, that the full therapeutic effect does not necessarily occur by administration of one dose, and may occur only after administration of a series of doses. Thus, an effective amount may be administered in one or more administrations. In the context of therapeutic or prophylactic applications, the amount of active agent administered to the subject will depend on the type and severity of the disease or condition and on the characteristics of the subject, such as general health, age, sex, body weight and tolerance to drugs. It will also depend on the degree, severity and type of disease or condition. The skilled artisan will be able to determine appropriate dosages depending on these and other factors. The anti-Lck intrabody can also be administered in combination with one or more additional therapeutic compounds.

“Sample” or “biological sample” as used herein means a biological material isolated from an individual.

As used herein, “specific binding” refers to the binding of an antigen by an antibody with a dissociation constant (K_{d}) of about 1 μM or lower as measured, for example, by surface plasmon resonance (SPR).

As used herein, the term “individual” or “patient” or “subject” (as in the subject of the treatment) refers to any animal (e.g., a mammal), including, but not limited to humans, non-human primates, rodents, and the like. The individual is, in one embodiment, a human being. Typically, the terms “individual”, “subject” and “patient” are used interchangeably herein in reference to a human subject.

As used herein, a “normal subject” or “control subject” refers, depending on the context, to a subject not suffering from idiopathic pulmonary fibrosis (IPF).

As used herein, a “control sample” refers to a sample from a control subject or a sample representative of a population of control subjects.

An “antibody” (Ab) shall include, without limitation, an immunoglobulin which binds specifically to an antigen and comprises at least two heavy (H) chains and two light (L) chains interconnected by disulfide bonds, or an antigen-binding portion thereof. Each H chain comprises a heavy chain variable region (abbreviated herein as V_{II}) and a heavy chain constant region. The heavy chain constant region comprises three constant domains, C_{H1}, C_{H2} and C_{H3}. Each light chain comprises a light chain variable region (abbreviated herein as V_{L}) and a light chain constant region. The light chain constant region comprises one constant domain, C_{L}. The V_{H} and V_{L} regions can be further subdivided into regions of hypervariability, termed complementarity determining regions (CDRs), interspersed with regions that are more conserved, termed framework regions (FR). Each V_{H} and V_{L} comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen.

An “antigen binding portion” of an Ab (also called an “antigen-binding fragment”) or antigen binding portion thereof refers to one or more sequences of an Ab (full length or fragment of the full length antibody) that retain the ability to bind specifically to the antigen bound by the whole Ab. Examples of an antigen-binding fragment include intrabody, Fab, F(ab′)₂, scFv (single-chain variable fragment), Fab′, dsFv, sc(Fv)2, and scFv-Fc.

As used herein, the term “variable domain” refers to immunoglobulin variable domains defined by Kabat et al., Sequences of Immunological Interest, 5th ed., U.S. Dept. Health & Human Services, Washington, D.C. (1991). The numbering and positioning of CDR amino acid residues within the variable domains is in accordance with the well-known Kabat numbering convention. VH, “variable heavy chain” and “variable heavy chain domain” refer to the variable domain of a heavy chain. VL, “variable light chain” and “variable light chain domain” refer to the variable domain of a light chain.

A “humanized” antibody refers to an Ab in which some, most or all of the amino acids outside the CDR domains of a non-human Ab are replaced with corresponding amino acids derived from human immunoglobulins. In one embodiment of a humanized form of an Ab, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the Ab to bind to a particular antigen. A “humanized” Ab retains an antigenic specificity similar to that of the original Ab.

An “intrabody” refers to a recombinant antibody that functions inside of a cell. An intrabody may affect target function by various mechanisms, such as interfering with enzymatic function and/or blocking protein-protein interactions. An intrabody can be an scFv. Intrabodies are engineered to be retained intracellularly, e.g., retained in the cytoplasm. Vectors that can target intrabodies are commercially available and known in the art. Expression of an intrabody can be regulated through the use of inducible promoters in the nucleic acid vector that encode the intrabody. Methods of making an intrabody are known in the art (see, e.g., Biocca, 2011, Antibody Expression and Production, 7:179-195 and Marschall et al, 2015, mAbs, 7(6): 1010-1035).

The anti-Lck antibody such as an anti-Lck intrabody of the present disclosure comprises a variable heavy chain and a variable light chain, each of which contains three complementarity-determining regions (CDRs) and four framework regions (FRs), arranged from amino-terminus to carboxy-terminus in the following order: FR1, CDR1, FR2, CDR2, FR3, CDR3, FR4. The CDRs contain most of the residues that form specific interactions with the antigen and are primarily responsible for antigen recognition. In other aspects, the anti-Lck antibody such as an anti-Lck intrabody comprises a single variable domain antibody, e.g., a single domain antibody (sdAb), also known as nanobody or camelid Vial antibody. Such antibodies have a single variable domain (a variable heavy chain V_{H} or a variable light chain V_{L}).

Biological samples useful in the practicing the methods of the invention are biological samples of fluids or tissues. An exemplary biological tissue is lung tissue. Exemplary biological fluids are blood, serum, plasma, and bronchoalveolar lavage (BAL) fluid. The biological sample is obtained from the subject using conventional methods known in the art.

The methods of the disclosure may be carried out with any subject. The subject is preferably a mammal, more preferably a primate and more preferably still, a human. In some embodiments, the subject has or is suspected of having IPF. In other embodiments, the methods are carried out with a subject at risk of developing IPF. Risk factors for IPF include occupational exposure and environmental pollution such as metals, animals, wood chips, cigarette smoking, gastroesophageal reflux disease, inflammatory bowel disease, Crohn's disease, smog, male sex, and age of 50 years or older.

To “treat” a disease as the term is used herein, means to reduce the frequency or severity of at least one sign or symptom of a disease or disorder experienced by a subject. Treating may include the postponement of further disease progression, or reduction in the severity of symptoms that have or are expected to develop, ameliorating existing symptoms and preventing additional symptoms.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

As envisioned in the present invention with respect to the disclosed compositions of matter and methods, in one aspect the embodiments of the invention comprise the components and/or steps disclosed herein. In another aspect, the embodiments of the invention consist essentially of the components and/or steps disclosed herein. In yet another aspect, the embodiments of the invention consist of the components and/or steps disclosed herein.

### DETAILED DESCRIPTION

Embodiments of the present invention are described below. It is, however, expressly noted that the present invention is not limited to these embodiments, but rather the intention is that modifications that are apparent to the person skilled in the art and equivalents thereof are also included.

T lymphocytes, one of the major inflammatory cell types, accumulate and release pro-fibrotic mediators (Wollin et al, 2019, Eur Respir J. 54(3); Luzina et al, 2008, J Leukoc Biol 83(2): 237-244). Progressive fibroblast proliferation and differentiation to myofibroblasts result in increased tissue stiffness leading to fibrotic scarring. The release of pro-fibrotic mediators by T cells stimulates fibroblast proliferation and differentiation to myofibroblasts (Petrasca et al., 2020, Rheumatology (Oxford) 2020 Feb. 11. pii: kez682. doi:10.1093/rheumatology/kez682. [Epub ahead of print]; Cope et al., 2007, Clin Exp Rheumatol 25(5 Suppl 46): S4-11; O'Reilly et al., 2012, Rheumatology (Oxford) 51(9): 1540-1549). We have reported impaired lung regeneration in patients with IPF (Moimas et al., 2019, ERJ Open Res. 2019 Dec. 16; 5(4):00138-2019).

“Idiopathic pulmonary fibrosis” or “IPF” refers to a disease in which scarring (fibrosis) of the lungs occurs for an unknown reason. The disease progresses with continued scarring of the lungs, and decreased oxygen uptake. IPF may be diagnosed using different tests including pulmonary function test (PFT), six-minute walk test, chest x-ray, blood tests, computed tomography (CT scan), bronchoscopy, and surgical lung biopsy. Risk factors for IPF include occupational exposure and environmental pollution such as metals, animals, wood chips, cigarette smoking, gastroesophageal reflux disease, inflammatory bowel disease, Crohn's disease, smog, male sex, and age of 50 years or older.

Lymphocyte-specific protein tyrosine kinase (Lck) is a member of the Src kinase family. Lck is associated with cytoplasmic domains of CD4 and CD8 receptors on CD4+ and CD8+ T cells, respectively. The structure of Lck is comprised of three domains, one SH3 in tandem with a SH2 domain at the amino terminal, and the kinase domain at the carboxy terminal. Other names for Lck include tyrosine-protein kinase Lck, leukocyte C-terminal Src kinase (LSK), lymphocyte cell-specific protein-tyrosine kinase, Protein YT16, proto-oncogene Lck, T cell-specific protein-tyrosine kinase, and p56-LCK.

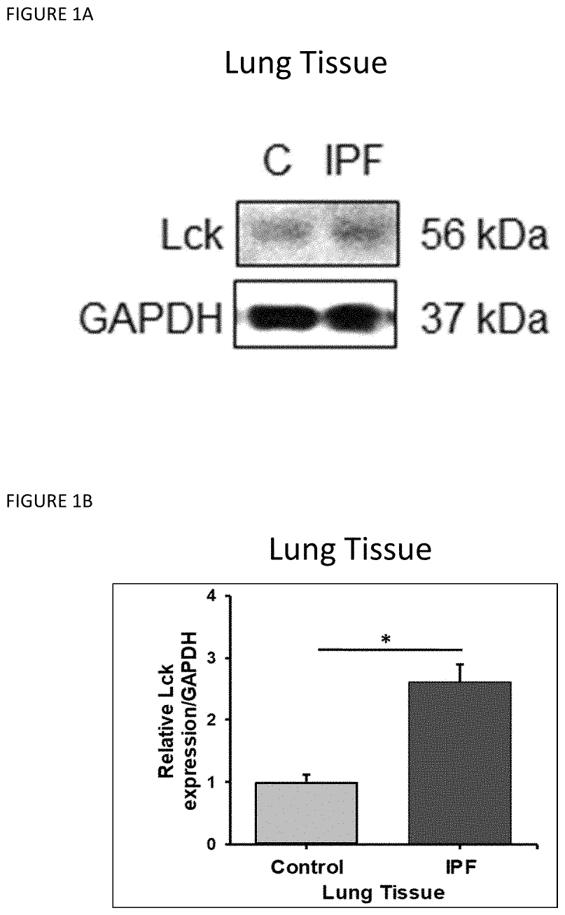

As shown herein, statistically significant higher Lck levels were observed in lung tissue obtained from patients with IPF (idiopathic pulmonary fibrosis) compared to controls (FIGS. 1A and 1B). In addition, it is considered possible that Lck contributes to a profibrotic T cell function. Targeting Lck to achieve selective suppression of T cell-mediated profibrotic response in IPF is an attractive clinical target. Profibrotic factors are known in the art and include, for instance, transforming growth factor-beta, tumour necrosis factor-alpha, platelet-derived growth factor, insulin-like growth factor-binding protein-5, and IL-6.

The disclosure provides an anti-Lck antibody. In some aspects, the anti-Lck antibody can be an intrabody. The anti-Lck intrabody contemplated by in the present disclosure specifically binds Lck in T cells and thereby inhibits secretion of at least one profibrotic factor. In an embodiment, the anti-Lck intrabody inhibits the interaction of Lck with CD4 receptor and/or CD8 receptor. In an embodiment, the anti-Lck intrabody inhibits the tyrosine kinase activity of Lck. In an embodiment, the anti-Lck intrabody inhibits interaction with the SH2 and/or SH3 domain of Lck.

In an embodiment, the anti-Lck intrabody specifically binds human Lck. Exemplary amino acid sequences of human Lck are: GenBank Accession No. AAH13200 (SEQ ID NO: 1) and AAC50287.1 (SEQ ID NO: 2) and naturally-occurring isoforms thereof. The anti-Lck intrabody may be an scFv prepared from a monoclonal antibody that specifically binds Lck, such as human Lck, using conventional methods in the art. The anti-Lck intrabody may be a humanized scFv. The technologies for scFv construction and humanization of antibody sequences, such as framework regions, have been well established.

Anti-Lck antibodies are known in the art. For instance, anti-Lck antibodies that recognize human Lck are commercially available from vendors including Invitrogen, Biolegend, Abcam, and BioRad.

The disclosure contemplates therapeutic administration of the anti-Lck intrabody to obtain a therapeutic effect for the treatment of IPF. A method of treating idiopathic pulmonary fibrosis (IPF) in a subject in need thereof is provided. The method comprises administering to the subject an effective amount of an anti-Lck intrabody that specifically binds Lck and thereby inhibits secretion of at least one profibrotic factor.

Therapeutic effect in the treatment of IPF can be measured as a reduction in the rate of decline of a pulmonary function parameter, such as a reduction by at least about 2%, at least about 5%, at least about 7% or great. Pulmonary function parameters include forced vital capacity (FVC), forced expiratory volume (FEV), vital capacity (VC), residual volume (RV), forced vital capacity percent (FVC %) predicted, forced expiratory flow (FEF), peak expiratory flow rate (PEFR), inspiratory reserve volume (IRV), functional residual capacity (FRC), inspiratory capacity (IC), total lung capacity (TLC), expiratory reserve volume (ERV), tidal volume (TV), and maximum voluntary ventilation (MVV). Therapeutic effect may also be measure by an increase in blood-oxygen saturation, an increase in progression-free survival, increased time to disease progression, a reduction in decline of carbon monoxide diffusion capacity (DLCO), and reduced lung fibrosis.

In an embodiment, administration is by transfection of target cells with an Adeno-associated virus (AAV) recombinantly engineered to comprise a coding sequence for the anti-Lck intrabody. The engineered AAV will be used for T cell infection with the anti-Lck intrabody, using methods known in the art. The technologies for engineering AAV to express a polypeptide and for transfecting cells with such AAV have been well established. In an embodiment, T cells of a patient diagnosed IPF are transfected with an engineered AAV for intracellular expression of the anti-Lck intrabody.

Combination therapy is also contemplated for treatment of IPF. In an embodiment, therefore, the method further comprises administration of at least one additional therapeutic agent. Exemplary therapeutics for combination therapy include, for instance, pirfenidone (Esbriet), nintedanib (OFEV), azathioprine, and N-acetylcysteine.

Further provided is a method for inhibiting fibroblast proliferation, fibroblast differentiation, and/or fibroblast activation by T cells in IPF in a subject in need thereof. The method comprises administering to the subject an effective amount of an anti-Lck intrabody that specifically binds Lck and thereby inhibits secretion of at least one profibrotic factor. In an embodiment, administration is by transfection of target cells with an Adeno-associated virus (AAV) recombinantly engineered to comprise a coding sequence for the anti-Lck intrabody.

Also provided is a method for analysis of Lck inhibition by intrabody in IPF.

Also provided is a method for screening or diagnosing for IPF comprising detecting the level of Lck in a test sample of a subject, comparing the level of Lck to the level of Lck in a control sample, wherein an elevated level of the Lck in the test sample as compared to the level in the control sample indicates the subject suffers from IPF. In some aspects, the level of Lck is detected by antibody binding. In some aspects, the control sample is from a healthy person without IPF. In some aspects, the control sample represents an average of Lck level detected in a group of healthy persons without IPF. In some aspects, the test sample and the control sample are the same type of biological tissue or fluid. In an aspect, the sample is lung tissue. In some embodiments, diagnosis of IPF is confirmed by lung biopsy and/or screening for other biomarkers for IPF, such as Krebs von den Lungen-6 (KL-6), leukocytes, and/or circulating innate immune cells. A subject diagnosed with IPF according to the method of the disclosure can be treated with a therapeutic agent for IPF, such as pirfenidone (Esbriet), nintedanib (OFEV), azathioprine, a corticosteroid such as prednisone, N-acetylcysteine, and any combination thereof. In some embodiments, a subject diagnosed with IPF can alternatively or additionally be treated with an anti-Lck intrabody.

In some embodiments, the IPF treatment comprises administering to the subject an effective amount of an anti-Lck intrabody that specifically binds Lck. Anti-Lck intrabody is expected to decrease the profibrotic T cell function. It is also expected that anti-Lck intrabody targeting CD4+ and CD8+ T cells will decrease an aggressive phenotype of fibroblasts resulting in a reduction of expression of key myofibroblast genes and their invasiveness in IPF. Exemplary myofibroblast genes in fibroblasts include, but are not limited to, α-SMA (α-smooth muscle actin), fibronectin, collagens (Col1a1 and Col3a1), periostin, and platelet-derived growth factor receptor alpha (PDFR). In some embodiments, the IPF treatment comprises administering at least one therapeutic agent chosen from pirfenidone (Esbriet), nintedanib (OFEV), azathioprine, and N-acetylcysteine. In some embodiments, treatment comprises both administering to the subject an effective amount of an anti-Lck intrabody that specifically binds Lck and at least one therapeutic agent chosen from pirfenidone (Esbriet), nintedanib (OFEV), azathioprine, N-acetylcysteine, or combinations thereof.

Assay methods used in the practice of the invention may utilize a substance comprising a binding agent that is specific for Lck, such as human Lck. Binding agents can be prepared, using conventional methods in the art, to bind specifically to Lck, such as human Lck, disclosed elsewhere herein.

In some embodiments, the methods of the invention can be carried out by detection of Lck inhibition by intrabody in a biological sample conventional methods in the art. Examples of such methods include, but is not limited to, Western blot analysis, immunoprecipitation, radioimmunoassay (RIA), immunofluorescent assay, chemiluminescent assay, flow cytometry, immunocytochemistry, enzyme-linked immunosorbent assay (ELISA), agglutination, precipitation, complement-fixation, and fluorescent antibodies. Assays based on protein-specific biomolecule interaction include, but are not limited to, antibody-based assays, aptamer-based assays, receptor and ligand assays, enzyme activity assays, and allosteric regulator binding assays. The invention is not limited to any one method of protein detection, but rather encompasses all presently known or heretofore unknown methods, such as methods that are discovered in the art. Proteins may be detected by other methods, e.g., mass spectroscopy analysis, that do not rely on a binding moiety.

The methods for detecting Lck in a biological sample can be readily adapted to kit form. A kit may contain a set of reagents that specifically detects for detection of Lck and one or more reagents and/or diluents for facilitating the contact of the reagent and Lck in the sample. The kit may further comprise instructions for using the kit according to the present invention. The reagent may comprise, for example, an antibody or aptamer specific for Lck. The antibody or aptamer may be detectably labeled, as described above. The kit can further comprise components necessary for detecting the detectable label (e.g., an enzyme or a substrate), and instrumentation for detection and measurement.

The kit can comprise, for example: (1) a first antibody (e.g., attached to a solid support) that binds to a marker protein; and, optionally, (2) a second, different antibody that binds to either the protein or the first antibody and is conjugated to a detectable label.

The kit may further comprise one or more buffers and/or reagents, e.g., labeling buffer and/or reagents, and detection means for detecting a detectable label. Protocols for using these buffers and reagents for performing different steps of the procedure may also be included in the kit.

The instructional material may comprise a publication, a recording, a diagram, or any other medium of expression which can be used to communicate the usefulness of the method. The package insert may comprise text housed in any physical medium, e.g., paper, cardboard, film, or may be housed in an electronic medium such as a diskette, chip, memory stick or other electronic storage form. The instructional material of the kit of the invention may, for example, be affixed to a container which contains other contents of the kit, or be shipped together with a container which contains the kit. Alternatively, the instructional material may be shipped separately from the container with the intention that the instructional material and the contents of the kit be used cooperatively by the recipient.

Provided also is an anti-Lck intrabody the specifically binds Lck in T cells and thereby inhibits secretion of at least one profibrotic factor for use in the treatment of IPF. Further provided is an anti-Lck intrabody the specifically binds Lck in T cells and thereby inhibits secretion of at least one profibrotic factor for use in the preparation of a medicament for the treatment of IPF. Also provided is a medicament for use in treating IPF in a subject, containing an anti-Lck intrabody that specifically binds Lck and thereby inhibits thereby inhibits secretion of at least one profibrotic factor from T cells.

Further provided is an anti-Lck intrabody the specifically binds Lck in T cells and thereby inhibits secretion of at least one profibrotic factor for use in inhibiting fibroblast proliferation, fibroblast differentiation, and/or fibroblast activation in a subject. Further provided is an anti-Lck intrabody the specifically binds Lck in T cells and thereby inhibits secretion of at least one profibrotic factor for use in the preparation in the preparation of a medicament for use in inhibiting fibroblast proliferation, fibroblast differentiation, and/or fibroblast activation in a subject. Also provided is a medicament for use in inhibiting fibroblast proliferation, fibroblast differentiation, and/or fibroblast activation in a subject, containing an anti-Lck intrabody that specifically binds Lck and thereby inhibits thereby inhibits secretion of at least one profibrotic factor from T cells.

### EXEMPLARY EMBODIMENTS

Among the embodiments provided herein are:

1. An anti-Lck intrabody that specifically binds Lck and thereby inhibits secretion of at least one profibrotic factor.
2. The anti-Lck intrabody of embodiment 1, wherein the anti-Lck intrabody inhibits the interaction of Lck with CD4 receptor and/or CD8 receptor.
3. The anti-Lck intrabody of embodiment 1, wherein the anti-Lck intrabody inhibits the tyrosine kinase activity of Lck.
4. The anti-Lck intrabody of embodiment 1, wherein the anti-Lck intrabody inhibits binding to the SH2 and/or SH3 domain of Lck.
5. A method of treating idiopathic pulmonary fibrosis (IPF) in a subject in need thereof comprising administering to the subject an effective amount of an anti-Lck intrabody that specifically binds Lck and thereby inhibits secretion of at least one profibrotic factor.
6. The method of embodiment 5, wherein the anti-Lck intrabody inhibits the tyrosine kinase activity of Lck.
7. The method of embodiment 5, wherein the anti-Lck intrabody inhibits binding to the SH2 and/or SH3 domain of Lck.
8. The method of embodiment 5, wherein the anti-Lck intrabody inhibits binding to the SH2 and/or SH3 domain of Lck.
9. The method of any one of embodiments 5-8, wherein the administration is by transfecting T cells of the subject with a recombinantly engineered Adeno-associated virus (AAV) comprising an expression vector comprising a coding sequence for the anti-Lck intrabody.
10. The method of anyone of embodiments 5-9, further comprising administration of at least one additional therapeutic agent.
11. The method of embodiment 10, wherein the at least one additional therapeutic agent is chosen from pirfenidone (Esbriet), nintedanib (OFEV), azathioprine, and N-acetylcysteine.
12. A method for inhibiting fibroblast proliferation, fibroblast differentiation, and/or fibroblast activation by T cells in a subject in need thereof comprising administering to the subject an effective amount of an anti-Lck intrabody that specifically binds Lck and thereby inhibits secretion of at least one profibrotic factor.
13. The method of embodiment 12, wherein the anti-Lck intrabody inhibits the tyrosine kinase activity of Lck.
14. The method of embodiment 12, wherein the anti-Lck intrabody inhibits binding to the SH2 and/or SH3 domain of Lck.
15. The method of embodiment 12, wherein the anti-Lck intrabody inhibits binding to the SH2 and/or SH3 domain of Lck.
16. The method of any one of embodiments 12-15, wherein the administration is by transfecting T cells of the subject with a recombinantly engineered Adeno-associated virus (AAV) comprising an expression vector comprising a coding sequence for the anti-Lck intrabody.
17. A method for screening for or diagnosing idiopathic pulmonary fibrosis (IPF) comprising detecting the level of Lck in a test sample of a subject, comparing the level of Lck to the level of Lck in a control sample, wherein an elevated level of the Lck in the test sample as compared to the level in the control sample indicates the subject suffers from IPF.
18. The method of embodiment 17, wherein the test sample is biological tissue or fluid selected from lung tissue, blood, serum, plasma, or bronchoalveolar lavage (BAL) fluid.
19. The method of embodiment 18, wherein the test sample is lung tissue.
20. The method of embodiment 17, 18 or 19, wherein the subject diagnosed with IPF is treated with a therapeutic agent for IPF.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### EXAMPLES

### Example 1: Lck Expression in Lungs of IPF Patients

We found statistically significantly higher Lck protein levels were detected by Western blotting in lung tissue obtained from IPF patients compared to controls lung tissue. See FIGS. 1A and 1B.

### Example 2: Anti-Lck Intrabody

An anti-Lck intrabody will be developed to specifically target CD4+ and CD8+ T cells and block secretion of profibrotic factors. We will use a single-chain variable fragment (scFv) with a size of 27 kDa, which is suitable for generating fusion proteins for efficient cellular expression. Adeno-associated virus (AAV) will be used for T cell infection with the anti-Lck intrabody.

An exemplary sequence for Lck for use in preparing an anti-antibody is: GenBank Accession No. AAC50287.1 (SEQ ID NO: 2) and naturally-occurring isoforms thereof. Another exemplary sequence for Lck for use in preparing an anti-antibody is: GenBank Accession No. AAH13200 (SEQ ID NO: 1) and naturally-occurring isoforms thereof. Upon development of at least one anti-Lck monoclonal antibody with suitable activity, using conventional methods in the art, an intrabody will be engineered using methods known in the art. The technologies for scFv construction and humanization of antibody sequences, such as framework regions, have been well established.

Adeno-associated virus (AAV) will be recombinantly engineered to comprise a coding sequence for the anti-Lck intrabody. The engineered AAV will be used for T cell infection with the anti-Lck intrabody, using methods known in the art. The technologies for engineering AAV to express a polypeptide and for transfecting cells with such AAV have been well established.

### Example 3: Determination of a Decrease of Profibrotic Mediators Secreted from CD4+ and CD8+ T Cells Obtained from Human IPF Lungs by Anti-Lck Intrabody

We will isolate T cells from control lungs obtained from organ donors, whose lungs are not suitable for transplantation and are donated for medical research through the Gift of Life Foundation (IRB protocol approved). Lungs from IPF transplants will be obtained from Temple University Hospital. CD4+ and CD8+ T cells will be isolated separately from the lung using CD4+ and CD8+ microbeads (Miltenyi Biotec Inc), respectively. T cells will be stimulated using anti-CD3 (1 μg/ml, Abcam) and anti-CD28 (0.5 μg/ml, Abcam). Anti-Lck intrabody will be delivered to the cell using AAV. The outcome measurement is T cell proliferation, assessed using carboxyfluorescein diacetate succinimidyl ester (Molecular Probes) by flow cytometry. Supernatant will be collected to determine cytokine and chemokine secretion by Luminex. We expect to detect higher CD4+ and CD8+ cell proliferation and secretion of profibrotic factors in IPF compared to controls. Anti-Lck intrabody is expected to decrease the profibrotic T cell function.

### Example 4: Determination of the Inhibition of Human Primary Pulmonary Fibroblasts Proliferation in IPF by Anti-Lck Intrabody

Fibroblasts will be co-cultured with CD4+ and CD8+ T cells treated with anti-Lck antibody using AAV. We will determine the effect of anti-Lck intrabody on CD4+ and CD8+ T cells by analysis of expression of the key myofibroblast genes in fibroblasts including α-SMA, fibronectin, collagens (Col1a1 and Col3a1), periostin, and platelet-derived growth factor receptor alpha by RT-PCR. Fibroblasts phenotype will be analyzed using cell contraction assay and invasion will be determined by transwell invasion assay. To measure cell proliferation, fibroblasts will be intracellularly labeled with Ki67 (BD Biosciences) and analyzed by immunofluorescence.

We expect that anti-Lck intrabody targeting CD4+ and CD8+ T cells will decrease an aggressive phenotype of fibroblasts resulting in a reduction of expression of key myofibroblast genes and their invasiveness in IPF. If determined, these data will confirm that the Lck-mediated reprogramming of CD4+ and CD8+ T cells can also promote lung regeneration in this disease.

The following pertains to Examples 3 and 4.

Study Design: Human samples will be randomly used for the study conditions. We will use the lungs obtained from both females and males with matched age and race. Investigators will not know the analyzed cohort until all data are finalized.

Statistical analysis: Data analysis will be performed using GraphPad Prism 5.0 and reported as means±s.e.m. of at least three independent assays. A p-value of less than 0.05 will be considered to indicate a significant difference.

Power analysis: The study includes lungs obtained from 7 control organ donors and 7 IPF patients. Assuming the probability of presence (or absence) of the outcome is 87.5% and 12.5%, respectively, then 7 subjects per group will achieve 87% power to detect a difference between the group proportions of 87.5%−12.5%=75%. The test statistic used is the two-sided Fisher's exact test. P-value was targeted at 0.05 and the significance level achieved by this design is 0.001.

The disclosures of each and every patent, patent application, GenBank record, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope used in the practice of the invention. The appended claims are intended to be construed to include all such embodiments and equivalent variations.

What is claimed is:

1. A method of treating idiopathic pulmonary fibrosis (IPF) in a subject in need thereof comprising administering to the subject an effective amount of an anti-Lck intrabody that specifically binds Lck and thereby inhibits secretion of at least one profibrotic factor.

2. The method of claim 1, wherein the anti-Lck intrabody inhibits the tyrosine kinase activity of Lck.

3. The method of claim 1, wherein the anti-Lck intrabody inhibits binding to the SH2 and/or SH3 domain of Lck.

4. The method of claim 1, wherein the anti-Lck intrabody inhibits binding to the SH2 and/or SH3 domain of Lck.

5. The method of claim 1, wherein the administration is by transfecting T cells of the subject with a recombinantly engineered Adeno-associated virus (AAV) comprising an expression vector comprising a coding sequence for the anti-Lck intrabody.

6. The method of claim 1, further comprising administration of at least one additional therapeutic agent.

7. The method of claim 6, wherein the at least one additional therapeutic agent is at least one of pirfenidone (Esbriet), nintedanib (OFEV), azathioprine, and N-acetylcysteine.

8. A method for inhibiting fibroblast proliferation, fibroblast differentiation, and/or fibroblast activation by T cells in a subject in need thereof comprising administering to the subject an effective amount of an anti-Lck intrabody that specifically binds Lck and thereby inhibits secretion of at least one profibrotic factor.

9. The method of claim 8, wherein the anti-Lck intrabody inhibits the tyrosine kinase activity of Lck.

10. The method of claim 8, wherein the anti-Lck intrabody inhibits binding to the SH2 and/or SH3 domain of Lck.

11. The method of claim 8, wherein the anti-Lck intrabody inhibits binding to the SH2 and/or SH3 domain of Lck.

12. The method of claim 8, wherein the administration is by transfecting T cells of the subject with a recombinantly engineered Adeno-associated virus (AAV) comprising an expression vector comprising a coding sequence for the anti-Lck intrabody.

13. A method for screening for or diagnosing idiopathic pulmonary fibrosis (IPF) comprising detecting the level of Lck in a test sample of a subject, comparing the level of Lck to the level of Lck in a control sample, wherein an elevated level of the Lck in the test sample as compared to the level in the control sample indicates the subject suffers from IPF.

14. The method of claim 13, wherein the test sample is biological tissue or fluid selected from lung tissue, blood, serum, plasma, or bronchoalveolar lavage (BAL) fluid.

15. The method of claim 13, wherein the subject diagnosed with IPF is treated with a therapeutic agent for IPF.

16. An anti-Lck intrabody that specifically binds Lck and thereby inhibits secretion of at least one profibrotic factor.

17. The anti-Lck intrabody of claim 16, wherein the anti-Lck intrabody inhibits the interaction of Lck with CD4 receptor and/or CD8 receptor.

18. The anti-Lck intrabody of claim 16, wherein the anti-Lck intrabody inhibits the tyrosine kinase activity of Lck.

19. The anti-Lck intrabody of claim 16, wherein the anti-Lck intrabody inhibits binding to the SH2 and/or SH3 domain of Lck.
